(19) United States
(12) Patent Application Publication
(10) Pub. No.: US 2024/0090615 A1
(43) Pub. Date: Mar. 21, 2024

(54) OPTIMIZED WOUND SITE OFFLOADING FOOTWEAR

(71) Applicants: TECHNION RESEARCH & DEVELOPMENT FOUNDATION LIMITED, Haifa (IL); RAMOT AT TEL-AVIV UNIVERSITY LTD., Tel-Aviv (IL)

(72) Inventors: Alon WOLF, Haifa (IL); Amit GEFEN, Tel Aviv (IL); Hadar SHAULIAN, Haifa (IL)

(21) Appl. No.: 18/038,534

(22) PCT Filed: Nov. 25, 2021
(86) PCT No.: PCT/IL2021/051408

§ 371 Date: May 24, 2023

Related U.S. Application Data
(60) Provisional application No. 63/118,682, filed on Nov. 26, 2020.

(51) Int. Cl. A43B 7/1455; A43B 7/144; A61B 5/103

(52) U.S. Cl. CPC A43B 7/147; A43B 7/144; A61B 5/1038

(57) ABSTRACT

A foot support structure having: a foot support surface for engagement by a foot of a subject; and a tissue loading alleviation zone within the foot support surface, such that the tissue loading alleviation zone defines a region of the foot support surface configured for optimizing an internal tissue loading state in a volume-of-interest (VOI) in said foot of said subject, and the region has at least one mechanical property value that gradually varies along at least one dimension of the region.

### CROSS REFERENCE TO RELATED APPLICATIONS

This application claims the benefit of priority under 35 U.S.C. § 119(e) of U.S. Provisional Patent Application No. 63/118,682, filed Nov. 26, 2020, entitled “OPTIMIZED WOUND SITE OFFLOADING FOOTWEAR”. The contents of the above applications are all incorporated by reference as if fully set forth herein in their entirety.

### BACKGROUND

This invention relates to the field of devices of treatment of foot conditions.

Diabetic foot ulceration is among the most common, serious and destructive complications of diabetes worldwide. Over the course of their disease, 25% of people with diabetes will develop a foot ulcer, the leading cause of lower extremity amputation in up to 80% of the cases. Lower extremity amputations are the costliest and most feared consequence of a foot ulcer, comprising up to one-third of the direct cost of diabetes care. Retrospective and prospective studies have shown that focal pressure and repetitive normal and shear stresses at a given location are the causative factors in the development of foot ulceration.

During gait, the repetitive large external normal and shear reaction forces cause elevated local mechanical loading on the foot, leading to an increased risk of foot ulcer development. Therefore, optimal offloading of the foot ulcer locations, while minimizing loads redistributed to peripheral tissues, is an essential component in preventing and treating foot ulcers.

Loading alleviation is often aided by the use of standard therapeutic shoes or custom-made insoles to offload high internal and/or plantar loading and to accommodate foot deformities. One specific common medical offloading device is a custom-made insole designed with a hole under the active wound site, to reduce normal and shear stresses on the ulcer and redistributes them among other more peripheral foot regions.

The foregoing examples of the related art and limitations related therewith are intended to be illustrative and not exclusive. Other limitations of the related art will become apparent to those of skill in the art upon a reading of the specification and a study of the figures.

### SUMMARY OF THE INVENTION

The following embodiments and aspects thereof are described and illustrated in conjunction with systems, tools and methods which are meant to be exemplary and illustrative, not limiting in scope.

There is provided, in an embodiment, a foot support structure comprising: a foot support surface for engagement by a foot of a subject; and a tissue loading alleviation zone within the foot support surface, wherein the tissue loading alleviation zone defines a region of the foot support surface configured for optimizing an internal tissue loading state in a volume-of-interest (VOI) in the foot of the subject, and wherein the region has at least one mechanical property value that gradually varies along at least one dimension of the region and wherein said mechanical property's graduated variation is determined based on said internal tissue loading state.

In some embodiments, the internal tissue loading state is determined based on one or more of: tissue strain, tissue compressive strain, tissue tensile strain, tissue shear strain, tissue strain energy density, tissue compressive stress, tissue tensile stress, tissue shear stress, and tissue hydrostatic pressure.

In some embodiments, the at least one mechanical property is one of: resiliency, flexibility, elasticity, density, stiffness, and compressibility.

In some embodiments, the region comprises a plurality of sub-zones, and wherein the at least one mechanical property value gradually varies by associating each of the sub-zones with a specified value of the at least one mechanical property.

In some embodiments, the optimizing comprises determining, with respect to the tissue loading alleviation zone, one or more of: dimensions of the tissue loading alleviation zone, an outline of the tissue loading alleviation zone, arrangement of the sub-zones within the tissue loading alleviation zone, a number of the sub-zones, dimensions of each of the sub-zones, and at least one mechanical property value associated with each of the sub-zones.

In some embodiments, the sub-zones are arranged in a sequence representing a graduated change in the at least one mechanical property along the at least one dimension of the region.

In some embodiments, the graduated change represents one of: an increase or a decrease in the value of the at least one mechanical property along the at least one dimension of the region.

In some embodiments, the foot support structure comprises a plurality of sections, wherein each of the sections may be removably secured to the foot support structure, and wherein all of the sections together form the foot support surface for engagement by the foot of the subject.

In some embodiments, each of the sections is associated with a specified value of the at least one mechanical property.

In some embodiments, the plurality of sub-zones comprises a plurality of the sections arranged to create the tissue loading alleviation zone based, at least in part, on the associated at least one mechanical property of each of the sections.

In some embodiments, the plurality of sub-zones comprises a plurality of circumscribed rings, and wherein each of the plurality of circumscribed rings is associated with a specified value of the at least one mechanical property.

In some embodiments, the graduated change represents one of: an increase or a decrease in the value of the at least one mechanical property from an outermost one to an innermost one of the plurality of circumscribed rings.

In some embodiments, the at least one dimension comprises one of: from one side of the region to an opposite side of the region, along a length dimension of the region, along a width dimension of the region, from a periphery to a center of the region, and from the center to the periphery of the region.

In some embodiments, the VOI encompasses at least one of: an ulcerated region of the foot, a region of the foot representing ulceration risk, and a peripheral region surrounding an ulcerated region of the foot.

In some embodiments, the VOI comprises at least a high-risk sub-VOI and a peripheral sub-VOI.

In some embodiments, the high-risk sub-VOI represents one or more of: the ulcerated region of the foot, and the region of the foot representing ulceration risk.

In some embodiments, the peripheral sub-VOI represents a tissue volume of the foot surrounding the high-risk sub-VOI.

In some embodiments, the VOI is associated with a contact area of the foot with the foot support surface during a gait cycle.

In some embodiments, the foot support structure is any one of: an orthotic, a prosthetic, a footwear article, a shoe upper, a sole insert, a midsole, and an inner sole.

There is also provided, in an embodiment, a method comprising: receiving, as input, a volumetric scan of an anatomy of a foot of a subject; generating data representing an internal tissue loading state of a volume of interest (VOI) in the foot, based on the scan; defining, in the VOI, a high-risk sub-VOI and a peripheral sub-VOI; and calculating, based on the data, a set of parameters for a tissue loading alleviation zone within a foot support surface of a foot support structure for engagement by the foot of the subject, wherein the calculation minimizes a combined tissue loading index in the high-risk and peripheral sub-VOIs.

In some embodiments, the method further comprises using the set of parameters to generate a set of manufacturing instructions for a foot support structure for the subject.

In some embodiments, the VOI is associated with a contact area of the foot with the foot support surface during a gait cycle.

In some embodiments, the VOI encompasses at least one of: an ulcerated region of the foot, a region of the foot representing ulceration risk, and a peripheral region surrounding an ulcerated region of the foot.

In some embodiments, the high-risk sub-VOI represents one or more of: the ulcerated region of the foot, and the region of the foot representing ulceration risk.

In some embodiments, the peripheral sub-VOI represents a tissue volume of the foot surrounding the high-risk sub-VOI.

In some embodiments, the data representing an internal tissue loading state takes into account mechanical properties of at least some of: foot bone tissue, foot cartilage, foot tendons, foot soft tissue, and foot skin within the VOI.

In some embodiments, with respect to each of the high-risk sub-VOI and peripheral sub-VOI, the tissue loading represents one or more of: tissue strain, tissue compressive strain, tissue tensile strain, tissue shear strain, tissue strain energy density, tissue compressive stress, tissue tensile stress, tissue shear stress, and tissue hydrostatic pressure.

In some embodiments, the tissue loading alleviation zone represents a downward concavity in the pressure support surface, and wherein the set of parameters comprises one or more of: (i) dimensions of the tissue loading alleviation zone; (ii) an outline of a peripheral edge of the tissue loading alleviation zone; (iii) a total surface area defined by the peripheral edge; (iv) a depth of the tissue loading alleviation zone; and (v) a radius of curvature of the peripheral edge.

In some embodiments, the outline is circular, and the set of parameters comprises a diameter of the outline.

In some embodiments, the set of parameters gradually varies at least one mechanical property value along at least one dimension of a region defined by the tissue loading alleviation zone.

In some embodiments, the at least one mechanical property is one of: resiliency, flexibility, elasticity, density, stiffness, and compressibility.

In some embodiments, the region comprises a plurality of sub-zones, and wherein the at least one mechanical property value gradually varies by associating each of the sub-zones with a specified value of the at least one mechanical property.

In some embodiments, the sub-zones are arranged in a sequence representing a graduated change in the at least one mechanical property along the at least one dimension of the region.

In some embodiments, the graduated change represents one of: an increase or a decrease in the value of the at least one mechanical property along the at least one dimension of the region.

In some embodiments, the foot support structure comprises a plurality of sections, wherein each of the sections may be removably secured to the foot support structure, and wherein all of the sections together form the foot support surface for engagement by the foot of the subject.

In some embodiments, each of the sections is associated with a specified value of the at least one mechanical property.

In some embodiments, the plurality of sub-zones comprises a plurality of the sections arranged to create the tissue loading alleviation zone based, at least in part, on the associated at least one mechanical property of each of the sections.

In some embodiments, the plurality of sub-zones comprises a plurality of circumscribed rings, and wherein each of the plurality of circumscribed rings is associated with a specified value of the at least one mechanical property.

In some embodiments, the graduated change represents one of: an increase or a decrease in the value of the at least one mechanical property from an outermost one to an innermost one of the plurality of circumscribed rings.

In some embodiments, the at least one dimension comprises one of: from one side of the region to an opposite side of the region, along a length dimension of the region, along a width dimension of the region, from a periphery to a center of the region, and from the center to the periphery of the region.

In some embodiments, the data representing an internal tissue loading state is further based, at least in part, on data associated with gait analysis with respect to the subject.

In some embodiments, the data representing an internal tissue loading state uses a computational method selected from the group consisting of: finite element analysis, finite difference analysis, and finite volume analysis.

In some embodiments, the combined tissue loading index comprises a weighted operation of respective tissue loadings calculated with respect to the high-risk and the peripheral sub-VOIs.

In addition to the exemplary aspects and embodiments described above, further aspects and embodiments will become apparent by reference to the figures and by study of the following detailed description.

### BRIEF DESCRIPTION OF THE FIGURES

The present invention will be understood and appreciated more comprehensively from the following detailed description taken in conjunction with the appended drawings in which:

FIG. 1 illustrates an exemplary system 100 for optimizing tissue loading alleviation in patient-specific foot support structures, in accordance with some embodiments of the present invention;

FIG. 2 is an overview of the functional steps in a method for optimizing tissue loading alleviation in patient specific foot support structures, according to some embodiments of the present disclosure;

FIG. 3 is a flowchart detailing the functional steps in a process for generating data representing an internal tissue loading state of the present disclosure, according to some embodiments of the present disclosure;

FIG. 4 is a flowchart detailing the functional steps in a process for using data representing an internal tissue loading state to optimize tissue loading alleviation zone parameters for an offload device which optionally includes a graded stiffness configuration, according to some embodiments of the present disclosure;

FIG. 5A shows a volumetric scan of an anatomy of a foot of a subject;

FIG. 5B shows an anatomically realistic model of a VOI within a subject's foot, according to some embodiments of the present disclosure;

FIG. 6 shows data representing an internal tissue loading state may be generated with respect to an anatomically realistic model of a VOI within a subject's foot, according to some embodiments of the present disclosure;

FIG. 7A shows geometric parameters defining a downward concavity, including cavity radius (X), the radius of curvature (ROC) of the cavity descent (Y), and the depth of the cavity (Z), according to some embodiments of the present disclosure;

FIG. 7B shows defined sub-regions in a tissue loading alleviation zone of the present disclosure, e.g., circumscribed concentric or nonconcentric rings, according to some embodiments of the present disclosure;

FIG. 7C-7E show several embodiments of a tissue loading alleviation zone of the present disclosure comprising a plurality of defined sub-regions, according to some embodiments of the present disclosure;

FIG. 8 is a depiction of a comparison of states of mechanical loading in the skin and soft tissues of the heel, with five representative heel support configuration; flat support, SS=small offloading radius, small radius of curvature (ROC), SL=small offloading radius, large ROC, LS=large offloading radius, small ROC, LL=large offloading radius, large ROC. Offloading cavity depth of the four representative configurations was in the range of 9-14. (a) Plantar contact pressure distributions in bottom view. (b) Von-Mises stress distributions in cross section view. (c) Max shear stress distribution in cross section view;

FIG. 9 is a depiction of cumulative percentage of soft tissue exposure to von Mises stress with (a) Peripheral-VOI and (b) High-Risk-VOI when the heel was loaded with flat support and four representative offloading cavity supports; SS=small offloading radius, large ROC, SL=small offloading radius, large ROC, LS=large offloading radius, small ROC, LL=large offloading radius, large ROC;

FIG. 10 is a depiction of normalized von Mises TSCE values for the 150 offloading cavity support configurations of (a) Peripheral-VOI, (b) High-Risk-VOI. Each dot represents support configuration with x,y,z axes represent the three cavity parameters. The five representative heel support configuration are marked; flat support, SS=small offloading radius, small ROC, SL=small offloading radius, large ROC, LS=large offloading radius, small ROC, LL=large offloading radius, large ROC;

FIG. 11 is a depiction of offloading depth vs. normalized von Mises TSCE for (a) Peripheral-VOI (R-Sq.=0.89) and (b) High-Risk-VOI (R-Sq.=0.96). To examine the specific effect of the offloading depth, TSCE values of the offloading radius and the offloading ROC were averaged per each depth step;

FIG. 12 is a depiction of weighted operation of normalized von Mises Peripheral-TSCE and High-Risk-TSCE for each of the 150 offloading cavity support configurations. TSCE is presented in log scale while each dot represents support configuration with x,y,z axes represent the three geometry parameters; and

FIG. 13 is a depiction of TSCE values of flat and total contact insole (TCI) with and without graded stiffness (GS) for (a) Peripheral-VOI and (b) High-Risk-VOI. The framed numbers represents the percentage change from non-graded to graded configuration per each pair of results.

### DETAILED DESCRIPTION

Disclosed herein is a foot support device or structure having a tissue loading alleviation zone which defines a plurality of sub-regions representing a differential and/or graduated levels of resiliency and/or stiffness across the tissue loading alleviation zone. In some embodiments, the tissue loading alleviation zone may optionally or alternatively define a downward concavity having a specified geometry.

In some embodiments, the present foot support structure may be configured for reducing mechanical loads, e.g., an internal tissue loading state, in one or more volumes of interest in the foot encompassing an ulcerated region of the foot and/or a region of the foot representing ulceration risk, and redistributing the loads among other more peripheral foot regions and/or volumes. In some embodiments, tissue loading state may be determined internally of the one or more volumes of interest in the foot.

Also disclosed are a method, system, and computer program product for optimizing a set of parameters associated with a tissue loading alleviation zone in a foot support device of the present disclosure, e.g., using patient-specific parameters and mechanical load modeling.

In some embodiments, the present disclosure provides for a customized design of a foot support structure based on patient-specific measurements and data. In some embodiments, the present method is based on an analysis of a three-dimensional model of internal tissue loading of one or more volumes-of-interest generated from an input foot volumetric scan of an anatomy of a foot of a subject, as well as additional patient data comprising foot tissue mechanical data and foot gait analysis data.

By way of background, a common medical offloading device is a custom-made insole designed with a hole under an active wound site, to reduce mechanical loading on an ulcer and redistribute them among other more peripheral foot regions. The size, location and shape of the hole is typically subjectively determined by a practitioner, based on visual indication and experience, in the absence of established guidelines for optimal design of the offloading hole.

The most common geometry of the hole is “step-shaped,” with relatively sharp peripheral edges. However, there are valid concerns that an aperture applied around the wound base increases shear and vertical forces at the peripheries of the wound (the peri-wound tissues), which escalates the risk of developing secondary ulcers at stress-concentration areas, a phenomenon also known as the “edge-effect.”

Foot ulcers often start internally and progress outwards. Thus, taking into account the foot internal layer stresses in designing an offload device may improve clinical outcomes of diabetic ulcer treatment and prevention.

Accordingly, in some embodiments, the present disclosure provides for determining an optimized tissue loading alleviation zone within a foot support structure, or a surface of a foot-receiving device, wherein the optimizing minimizes mechanical load in a defined one or more volumes-of-interest (VOI) in the foot of the subject.

As used herein, ‘mechanical load,’ ‘load,’ state,’ and ‘stress’ may be used interchangeably to denote a value or set of values representing the mechanical load applied to a volume-of-interest in a foot during a gait cycle, comprising, but not limited to, strain, compressive strain, tensile strain, shear strain, strain energy density, compressive stress, tissue tensile stress, shear stress, hydrostatic pressure, or any combination thereof.

As used herein, ‘foot support structure’ and ‘foot support device’ may be used interchangeably to denote any foot-receiving device or part thereof which includes a foot support surface or portion, and/or any foot support surface within a foot-receiving device, e.g., any orthotic, prosthetic, shoe upper, footwear article, sole insert, midsole, inner sole, and/or any other foot-supporting surface which interacts with the foot when the foot support structure is in use.

The foot support surface or structure may be embedded in the sole structure of an article of footwear. Although the terms “foot support” and “footbed” may be used, the plantar surface of a foot and the footbed of a foot-receiving device need not be in direct contact with the foot support portions when the foot-receiving device is in use. Therefore, it will be appreciated that the foot support structure may support a foot with one or more layers of material or other structures separating the plantar surface of the foot and/or the footbed of the foot-receiving device from the foot support structure.

In some embodiments, the present disclosure provides for a foot support device or structure having a tissue loading alleviation zone, wherein a plurality of parameters define an optimized tissue loading alleviation zone determined based on patient-specific data, measurements, and modeling.

In some embodiments, a tissue loading alleviation zone of the present disclosure may refer to any cavity, indentation, hole and/or any other defined region within a foot support structure of the present disclosure which defines (i) a downward concavity having a specified geometry, (ii) multiple sub-regions having differential and/or graduated resiliency or stiffness across a defined region, and (iii) any combination of (i) and (ii). In some embodiments, the tissue loading alleviation zone is configured to decrease a load applied locally to a specified region of the foot, e.g., during use of foot support structure.

In some embodiments, the optimizing is based on a set of parameters associated with a geometry of the tissue loading alleviation zone, and/or a selection of materials for one or more sub-regions of the tissue loading alleviation zone, and/or a selection of material properties (e.g., resiliency, stiffness, flexibility, density, and the like) for one or more sub-regions of the tissue loading alleviation zone.

In some embodiments, the optimizing comprises computation of a set of parameters, for example, a set of tissue loading alleviation zone parameters, comprising one or more of:
Dimensions of the tissue loading alleviation zone,
an outline of a peripheral edge of the tissue loading alleviation zone,
a total surface area defined by the peripheral edge,
a depth (e.g., average depth, maximal depth) of the tissue loading alleviation zone, and
a radius of curvature of the peripheral edge.

In some embodiments, the optimizing comprises defining a plurality of sub-zones in the tissue loading alleviation zones, e.g., circumscribed rings and/or any other configuration, wherein each sub-zone is associated with a specified value of at least one mechanical property, e.g., resiliency, flexibility, elasticity, density, stiffness, and/or compressibility.

In some embodiments, the sub-zones of the present tissue loading alleviation zone may be arranged in a contiguous sequence representing a gradual change in the at least one mechanical property value across a region defined by the tissue loading alleviation zone. In some embodiments, the set of parameters includes variation in the at least one mechanical property. For example, the sub-zones of the present tissue loading alleviation zones may define a series of circumscribed rings, wherein one or more specified mechanical properties of each ring (e.g., resiliency) may be gradually increasing from the outermost to the innermost ring, and wherein the optimizing comprises defining one or more of the following parameters, to be included in the set of parameters:
Dimensions of the tissue loading alleviation zone,
number of circumscribed concentric or nonconcentric rings,
inner and outer diameter of each ring, and
a resiliency value of each ring.

In some embodiments, at least one mechanical property of any sub-zone of the plurality of sub-zones may be adjusted and/or determined by, e.g., a choice of material (e.g., rubber, foam, urethane, etc.), and/or by adjusting a property of a material (e.g., foam density).

In some embodiments, at least one mechanical property of any sub-zone of the plurality of sub-zones may be gradually and/or differentially tuned across a region defined by the tissue loading alleviation zone, such that the tissue loading alleviation zone represents a graduated change in the at least one mechanical property, e.g., gradually increasing or decreasing from one side to an opposite side of the region, along a length dimension of the region, along a width dimension of the region, from a periphery to the center of the region, or from the center to the periphery of the region.

In some embodiments, the number of sub-zones of the present tissue loading alleviation zone may be between, e.g., 2 and 15. For example, a tissue loading alleviation zones defining a series of circumscribed concentric or nonconcentric rings may comprise between 2 and 15 rings. In other embodiments, a greater number of sub-zones may be used. In some embodiments, the circumscribed rings may each have variable dimensions, e.g., width, etc.

In yet other embodiments, the tissue loading alleviation zone may define a region representing a gradual change in at least one mechanical property value across one or more dimensions or directions in the defined region. In such embodiments, the tissue loading alleviation zone is not divided into defined sub-zones, but rather comprises a single defined zone having at least one mechanical property value varies gradually, e.g., increasing or decreasing, across one or more dimensions in the defined region, e.g., resiliency, flexibility, elasticity, density, stiffness, and/or compressibility. In some embodiments, the at least one mechanical property may thus be gradually varied, e.g., from one side to an opposite side of the region, along a length dimension of the region, along a width dimension of the region, from a periphery to the center of the region, or from the center to the periphery of the region.

In yet other embodiments, the tissue loading alleviation zone may define a region representing a gradual change in at least one mechanical property value across one or more dimensions or directions in the defined region. In such embodiments, the foot support surface of the present disclosure may comprise one or more regions, or may comprise in its entirety, a modular structure having a plurality of contiguous separate removable sections, which together form a normally substantially smooth surface for engagement by the foot. Each section of these sections may be individually removable and/or replaceable, e.g., by being removed from the space where it would normally be located, and by inserting a replacement section in its stead. In some embodiments, a plurality of sections may be provided, wherein each section or a group of sections may be associated with a specified value of at least one mechanical property, e.g., resiliency, flexibility, elasticity, density, stiffness, and/or compressibility. Thus, by arranging these sections in a desired pattern, a tissue loading alleviation zone may be created comprising a contiguous sequence of these sections representing a gradual change in the at least one mechanical property value across a region defined by the tissue loading alleviation zone. In some embodiments, tissue loading alleviation zone comprising such an arrangement may be modified over time, e.g., by replacing and/or substituting one or more sections therein with replacement sections having different mechanical properties, and/or by changing a surface area, an outline, and/or any other geometric dimension of the tissue loading alleviation zone.

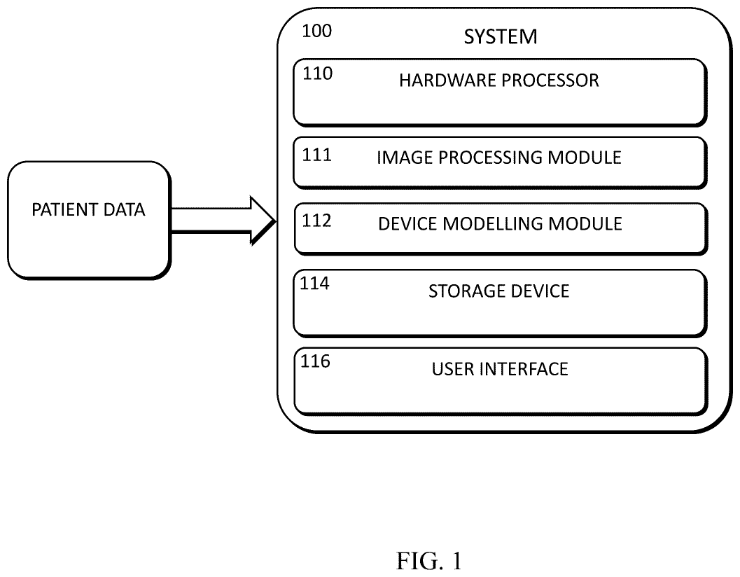

FIG. 1 illustrates an exemplary system 100 for optimizing tissue loading alleviation in patient-specific foot support structures, in accordance with some embodiments of the present invention.

System 100 as described herein is only an exemplary embodiment of the present invention, and in practice may have more or fewer components than shown, may combine two or more of the components, or a may have a different configuration or arrangement of the components. The various components of system 100 may be implemented in hardware, software, or a combination of both hardware and software. In various embodiments, system 100 may comprise a dedicated hardware device, or may form an addition to or extension of an existing device.

In some embodiments, system 100 may comprise a hardware processor 110 and memory storage device 114. In some embodiments, system 100 may store in a non-volatile memory thereof, such as storage device 114, software instructions or components configured to operate a processing unit (also “hardware processor,” “CPU,” “quantum computer processor,” or simply “processor”), such as hardware processor 110. In some embodiments, the software components may include an operating system, including various software components and/or drivers for controlling and managing general system tasks (e.g., memory management, storage device control, power management, etc.) and facilitating communication between various hardware and software components.

In some embodiments, non-transient computer-readable storage device 114 (which may include one or more computer readable storage mediums) is used for storing, retrieving, comparing, and/or annotating obtained volumetric scans and/or gait analysis data and/or tissue mechanical data. Scans may be stored on storage device 114 based on one or more attributes, or tags, such as a time stamp, a user-entered label, or the result of an applied image processing method indicating the association of the frames, to name a few.

The software instructions and/or components operating hardware processor 110 may include instructions for receiving and analyzing multiple scans captured by a suitable volumetric imaging system. For example, hardware processor 110 may comprise image processing module 111 and a device modeling module 112 implementing one or more three-dimensional (3D) tissue loading state analysis methods. Image processing module 110 receives a volumetric scan and optionally gait analysis and tissue mechanical data, and applies one or more image processing algorithms thereto. In some embodiments, image processing module 111 comprises one or more algorithms configured to perform object detection, classification, segmentation, and/or any other similar operation, using any suitable image processing, algorithm technique, and/or feature extraction process.

Device modeling module 112 may comprise one or more tissue loading state analysis implementations such as finite element methods implementations, and methods for combining tissue loading state scores of different VOI of foot models stored in storage device 114.

In some embodiments, system 100 may further comprise a user interface 116 comprising, e.g., a display monitor for displaying images, a control panel for controlling system 100, and a speaker for providing audio feedback.

As used herein, “volumetric imaging” or “volumetric scans” refer to techniques and processes for creating visual representations of internal anatomical structures throughout the human body, including bones, blood vessels, vertebrae, major organs, skin, etc. Volumetric imaging techniques include, among others, magnetic resonance imaging (MM), computed tomography (CT), ultrasound (US), positron emission tomography (PET), corrosion casting, micro-CT, electron microscopy, MR angiography and single photon emission tomography. Volumetric imaging techniques typically produce an image dataset comprising a series of two-dimensional (2D) cross-sectional ‘slice’ images acquired through the scanned volume. A three-dimensional (3D) volume can then be constructed from the image dataset.

As used herein, “image segmentation” refers to the process of partitioning an image into different meaningful segments, which may correspond to different tissue classes, organs, pathologies, and/or other biologically-relevant structures, for example through a binary classification of pixels or voxels in an image.

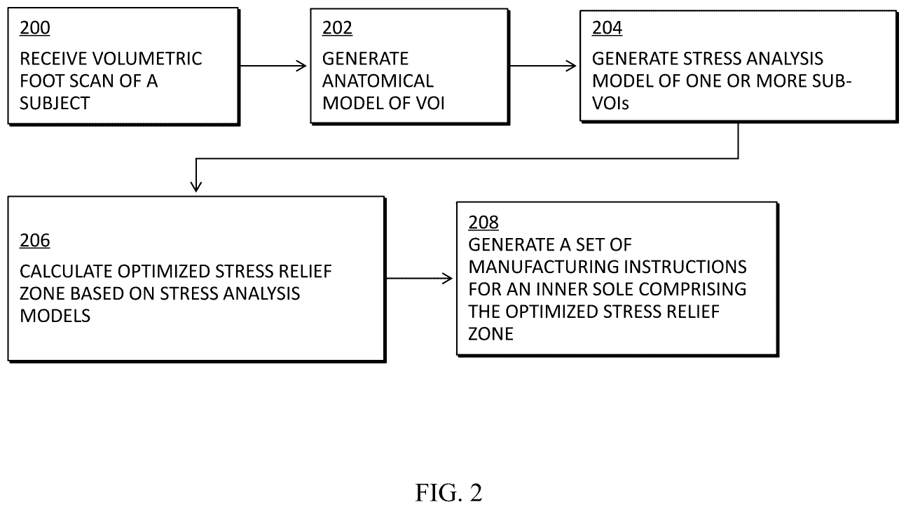

FIG. 2 is an overview of the functional steps in a method for optimizing tissue loading alleviation in patient-specific foot support structures, according to some embodiments of the present disclosure.

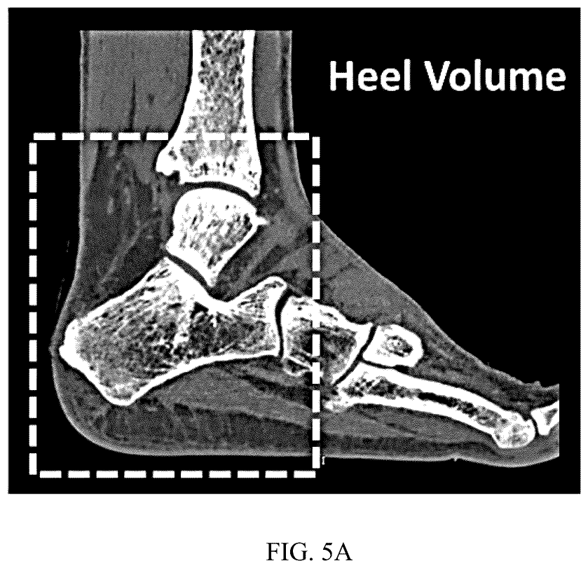

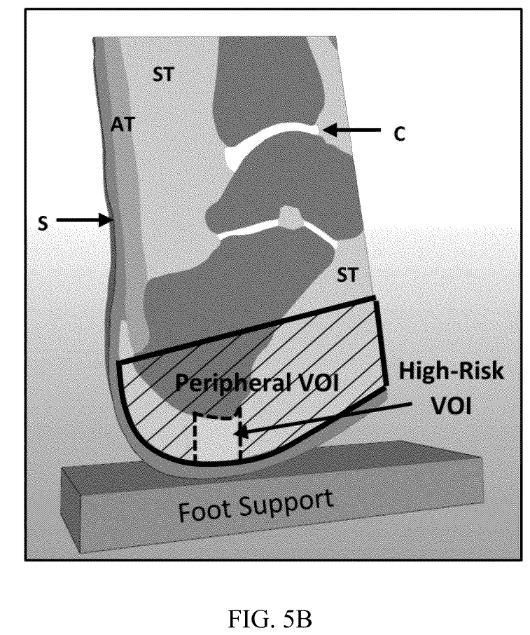

In some embodiments, at step 200, the present method provides for receiving a volumetric scan of an anatomy of a foot of a subject, for example, comprising a series of consecutive two-dimensional (2D) images of a foot or a specified region of a foot (e.g., heel, instep arch, ball, etc.) of a subject (see FIG. 5A) or a three-dimensional (3D) image of the foot or the specified region of the foot.

With reference to FIG. 5B, in some embodiments, at step 202, an anatomically realistic model of a VOI within the subject's foot may be generated from the 2D images or the 3D image. In some embodiments, the model includes at least some anatomical parts of the VOI (e.g., various bone portions, cartilage, tendons, soft tissue, and skin), as well as a contact area of the VOI with the ground during a gait cycle.

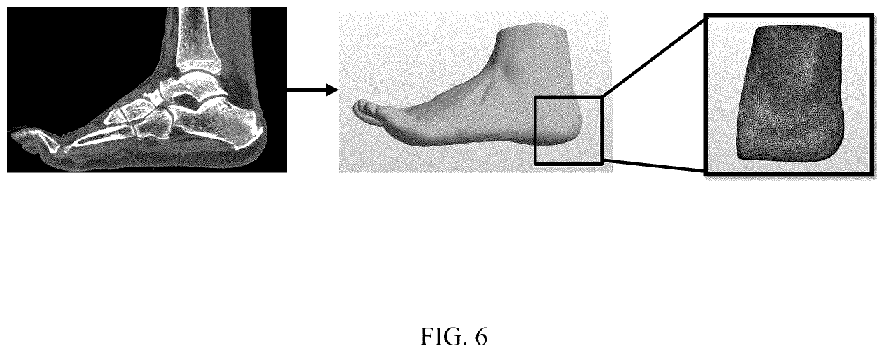

With reference to FIG. 6, in some embodiments, at step 204, data representing an internal tissue loading state may be generated with respect to the generated model. In some embodiments, the data representing an internal tissue loading state may reflect internal tissue loading state in two or more sub-VOIs which represent, e.g., (i) a high-risk sub-VOI comprising a damaged tissue area due to ulceration and/or an area having an increased likelihood of ulceration damage, and (ii) an area or volume surrounding and/or peripheral to the high-risk sub-VOI.

In some embodiments, the data representing internal tissue loading state may represent the mechanical load applied to the sub-VOIs, comprising, but not limited to, one or more of: strain, compressive strain, tensile strain, shear strain, strain energy density, compressive stress, tissue tensile stress, shear stress, hydrostatic pressure, and any combination thereof.

In some embodiments, the data representing an internal tissue loading state may be further based, at least in part, on data comprising, e.g., foot gait analysis data and/or data relating to foot tissue properties.

In some embodiments, the present disclosure uses computational methods, e.g., finite element (FE) analysis, finite difference analysis, finite volume analysis and the like, to construct the data representing an internal tissue loading state, wherein these methods provide for evaluation of internal tissue loads in a defined volume. This is contrary to methods using only peak foot plantar pressures or peak strains, which might lead to inaccurate results and biased conclusions, as the point loads are influenced by individual elements in the mesh, and which does not take into account the volumetric exposure of the tissue to the loading.

In some embodiments of the present invention, the data representing an internal tissue loading state is based on comprehensive FE modeling of a realistic foot region (e.g., heel), walking on various offloading support geometries, to compare the performance of various hole-support configurations and to determine the optimal offloading design of sole hole geometry. In some embodiments, the FE model targets optimal distribution and reduction of high heel loads to enable effective prevention and treatment of heel ulceration.

In some embodiments, at step 206, the data representing an internal tissue loading state is used to calculate a set of parameters for an optimized tissue loading alleviation zone to be implemented in, e.g., in a foot support structure of the present disclosure.

In some embodiments, the calculation of the set of parameters, may comprise calculating a set of geometric parameters for an optimized tissue loading alleviation zone, including, but not limited to:
dimensions and an outline of the tissue loading alleviation zone,
tissue loading alleviation zone surface area,
tissue loading alleviation zone maximal depth, and/or
a radius of curvature of a peripheral edge of the tissue loading alleviation zone.

In some embodiments, the calculation may be based on simulating multiple tissue loading alleviation zone configurations to arrive at an optimized combination of parameters which minimizes a combined tissue loading in the sub-VOIs.

Optionally or alternatively, the calculation may also provide for segmenting the tissue loading alleviation zone into two or more segments or regions of relative flexibility and relative stiffness. Accordingly, in some embodiments, the tissue loading alleviation zone may be segmented into two or more sub-zones, wherein each sub-zone may be associated with different material properties, e.g., stiffness, flexibility.

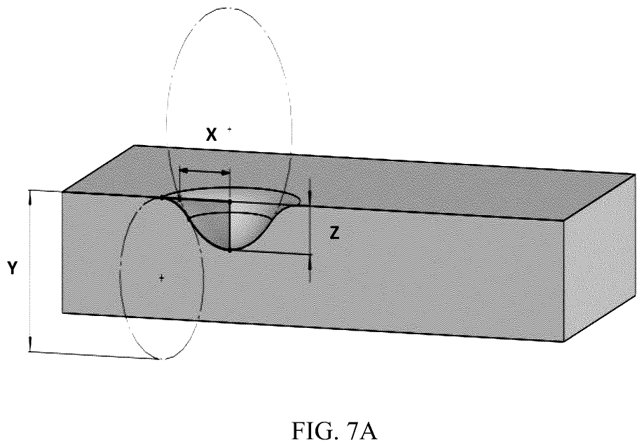

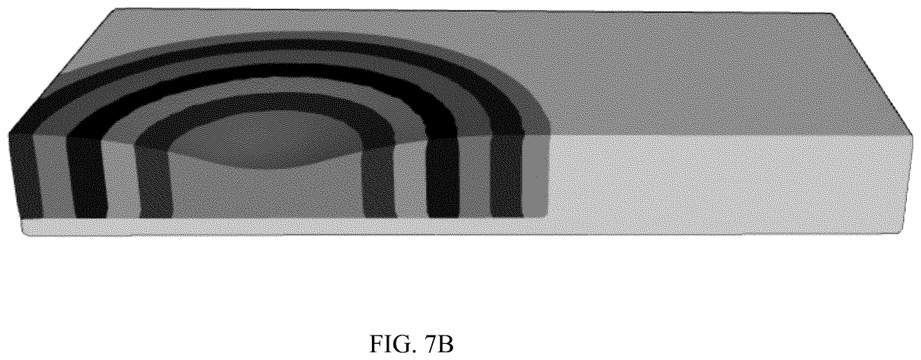

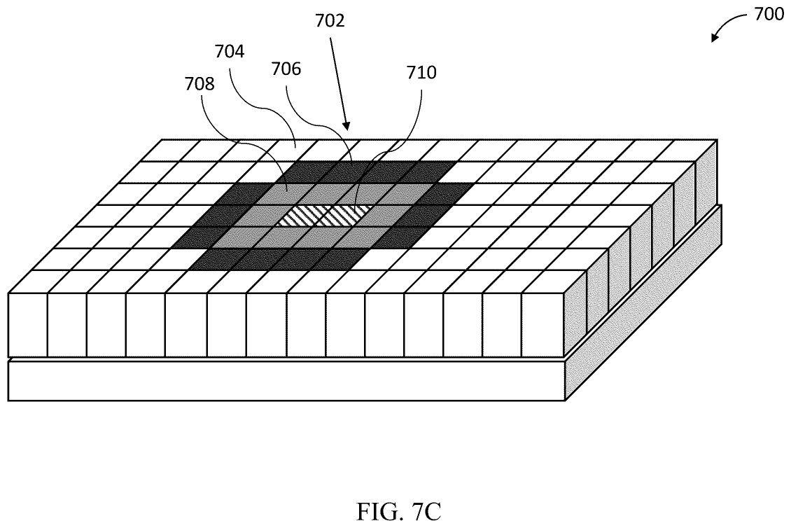

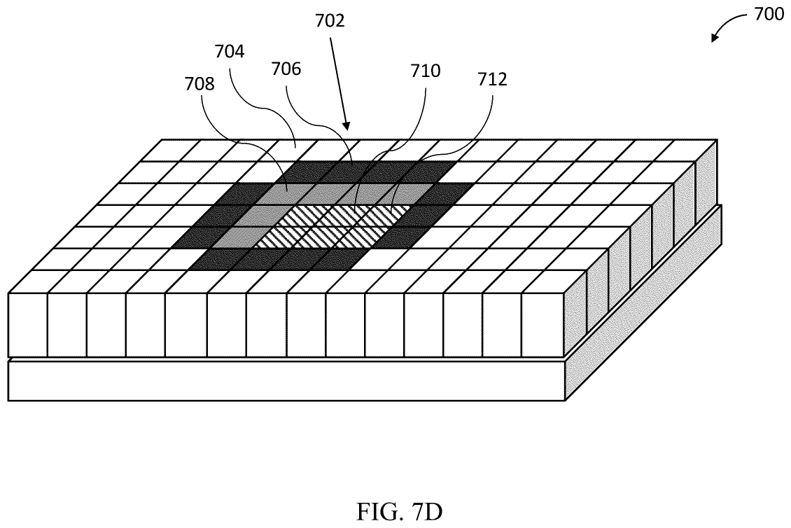

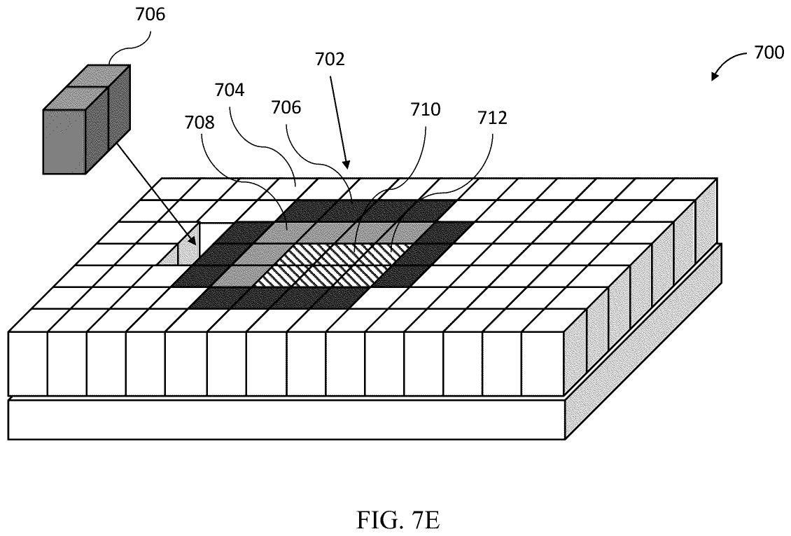

Accordingly, in some embodiments, the optimizing comprises defining a plurality of sub-zones in the tissue loading alleviation zones, e.g., circumscribed concentric or nonconcentric rings and/or any other configuration, wherein each sub-zone is associated with a specified value of at least one mechanical property, e.g., resiliency, flexibility, elasticity, density, stiffness, and/or compressibility. Nonlimiting examples for sub-zine are illustrated and discussed with respect to sections 704-710 of FIG. 7. In some embodiments, the sub-zones of the present tissue loading alleviation zone may be arranged so as to represent a gradual change in the at least one mechanical property value about a region defined by the tissue loading alleviation zone. In some embodiments, the at least one mechanical property value of any sub-zone of the plurality of sub-zones may be adjusted and/or determined by, e.g., a choice of material (e.g., rubber, foam, urethane, etc.), and/or by adjusting a property of a material (e.g., material density, thickness, etc.). In some embodiments, the at least one mechanical property value of any sub-zone of the plurality of sub-zones may be gradually and/or differentially modified or adjusted, with respect to a region defined by the tissue loading alleviation zone, e.g., from one side to an opposite side of the region, along a length dimension of the region, along a width dimension of the region, from the periphery to the center of the region, or from the center to the periphery of the region. For example, the sub-zones of the present tissue loading alleviation zones may define a series of circumscribed concentric or nonconcentric rings, wherein one or more specified mechanical properties of each ring (e.g., resiliency) may be gradually increasing (or decreasing) from the outermost to the innermost ring. In some embodiments, the mechanical property's graduated variation is determined based on the internal tissue loading state.

With reference to FIGS. 7C-7E, in some embodiments, a foot support surface 700 of the present disclosure may comprise a tissue loading alleviation zone 702 which may define representing a gradual change in at least one mechanical property value across one or more dimensions or directions in the defined region. In such embodiments, the foot support surface 700 of the present disclosure may comprise one or more regions, or may comprise in its entirety, a modular structure having a plurality of contiguous separate removable sections, e.g., sections 704-710, which together form a normally substantially smooth surface for engagement by the foot.

Thus, for example, a tissue loading alleviation zone 702 of the present disclosure may comprise an arrangement having plurality of individual sub-zones, e.g., high-stiffness section 704, medium-high stiffness section 706, medium stiffness sections 708, and low stiffness sections 710.

In some embodiments, each section of these sections 704-710 may be individually removable and/or replaceable, e.g., by being removed from the space where it would normally be located, and by inserting a replacement section in its stead, as shown in FIG. 7E. In some embodiments, a plurality of sections may be provided, wherein each section or a group of sections may form a sub-zone that may be associated with a specified value of at least one mechanical property, e.g., resiliency, flexibility, elasticity, density, stiffness, and/or compressibility. Thus, by arranging these sections in a desired pattern, a tissue loading alleviation zone may be created comprising a contiguous sequence of these sections representing a gradual change in the at least one mechanical property value across a region defined by the tissue loading alleviation zone. In some embodiments, tissue loading alleviation zone comprising such an arrangement may be modified over time, e.g., by replacing and/or substituting one or more sections therein with replacement sections having different mechanical properties, and/or by changing a surface area, an outline, and/or any other geometric dimension of the tissue loading alleviation zone.

Accordingly, in some embodiments, the optimizing thus comprises defining one of more of the following:
Dimensions of the tissue loading alleviation zone,
an outline of the tissue loading alleviation zone,
arrangement of sub-zones within the tissue loading alleviation zone (e.g., as circumscribed concentric or nonconcentric rings),
number of sub-zones (e.g., in the case of circumscribed rings, the number of rings),
dimensions of each sub-zone (e.g., in the case of circumscribed rings, inner and outer diameter of each ring), and
at least one mechanical property value of each sub-zone.

In some embodiments, the calculation may comprise both:
calculating a set of geometric parameters for an optimized tissue loading alleviation zone defining a downward concavity, and
calculating a value of one or more specified mechanical properties associated with each of a plurality of sub-zones in the tissue loading alleviation zones.

In some embodiments, at step 208, a set of manufacturing instructions may be generated with respect to a foot support structure of the present disclosure, incorporating the optimized tissue loading alleviation zone therein. In some embodiments, the manufacturing instructions may be generated with respect to any suitable manufacturing processes, e.g., molding, additive manufacturing (e.g., 3D printing), and the like.

A potential advantage of the present disclosure is that it provides for an optimized patient specific custom tissue loading alleviation design, which may improve treatment and prevention of ulcers symptomatic of various conditions such as diabetes.

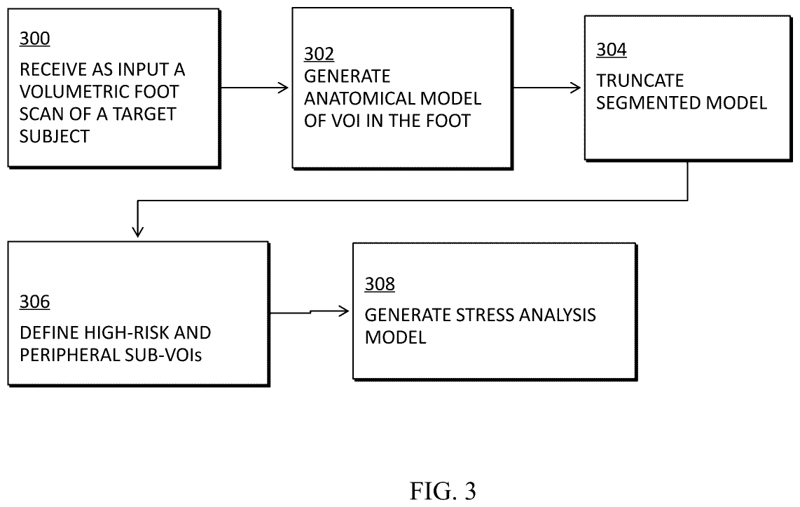

FIG. 3 is a flowchart detailing the functional steps in a process for generating data representing an internal tissue loading state of the present disclosure, as may be implemented by an embodiment of module 111 of system 100.

At step 300, a volumetric foot scan of an anatomy of a foot of a target subject is received. In a non-limiting example, the scan comprises a sequence of 2D scan slices or a 3D image (model). FIG. 5A shows a scan slice depicting a human heel.

At step 302, in some embodiments, an anatomically realistic model of a VOI within the subject's foot may be generated from the volumetric foot scan. In some embodiments, the model includes all anatomical parts of the VOI (e.g., various bone portions, cartilage, tendons, soft tissue, and skin), as well as a contact area of the VOI with the ground during a gait cycle.

In some embodiments, generating the anatomical model is based on segmenting, in the scan, the various anatomical constituents, e.g., bones, cartilage, tendons, soft tissue, and skin.

As can be seen in the non-limiting example of FIG. 5B which is an anatomically realistic model of a VOI within a subject's foot, in some embodiments, in the case of a heel region, the segmentation includes the heel bones (calcaneus bone, lower aspect of the tibia and fibula, and posterior aspect of the talus and cuboid), cartilage (C), Achilles Tendon (AT), soft tissue regions (ST) of the heel, and the skin (S).

In some embodiments, any suitable image processing tool may be used for generating the segmentation in the scan.

Next, at step 304, in some embodiments, the segmented VOI may be truncated in order to include the VOI relevant to optimizing the offload device (e.g. the contact area of the foot with the ground during heel strike).

At step 306, in some embodiments, the generated VOI may be divided into sub-VOIs, comprising, e.g., a high-risk sub-VOI and a peripheral sub-VOI.

With reference to FIG. 5B, the high risk sub-VOI comprises the bottom part of the heel soft tissue around the calcaneus soft tissue interface. The high-risk sub-VOI may represent damaged tissue due to ulceration under the sharpest point of the calcaneus bone, and/or soft tissue at high risk of ulceration, and defined as, e.g., a cylindrical area with skin projection area of approximately 2 cm², located in the soft tissue layer on the sole of the heel under the sharpest point at the surface of the calcaneus bone, where the highest interface pressures are usually measured on a flat support. The peripheral sub-VOI may be defined as the lower part of the heel soft tissue surrounding the high-risk area.

In some embodiments, at step 308, a data representing an internal tissue loading state may be generated with respect to the foot anatomical model shown in FIG. 5B, as detailed with reference to step 204 in FIG. 2.

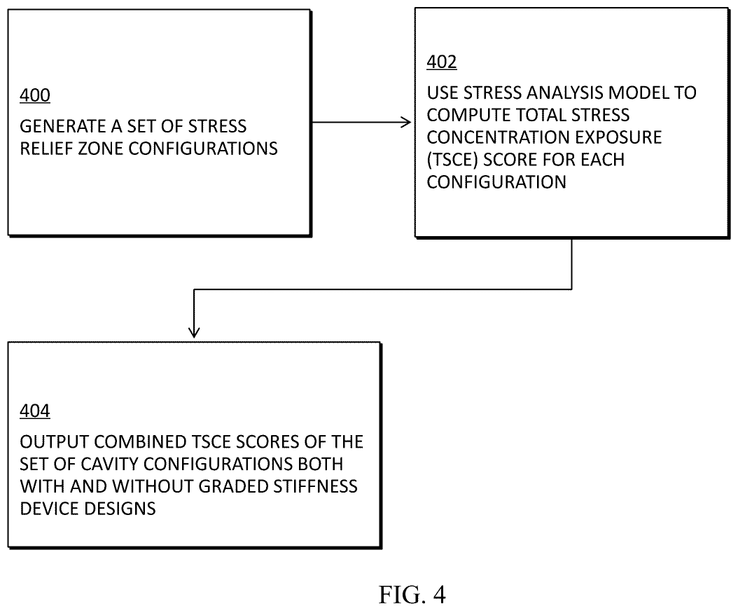

FIG. 4 is a flowchart detailing the functional steps in a process for using data representing an internal tissue loading state to optimize tissue loading alleviation zone parameters for a tissue loading alleviation device which optionally includes a graded stiffness configuration.

At step 400, in some embodiments, the present process is configured to generate a set of tissue loading alleviation zone candidate configurations, to simulate mechanical loads on a foot of a subject, using, e.g., the data representing an internal tissue loading state generated with reference to step 204 in FIG. 2.

In some embodiments, the set of tissue loading alleviation zone candidate configurations may include (i) geometric parameters defining a downward concavity, (ii) defined sub-regions in the tissue loading alleviation zones of relative material stiffness and flexibility, and/or (iii) any combination thereof.

In some embodiments, as can be seen in FIG. 7A, the geometric parameters defining a downward concavity include, but are not limited to:
dimensions and an outline of the tissue loading alleviation zone (X),
tissue loading alleviation zone surface area,
tissue loading alleviation zone maximal depth (Z), and/or
a radius of curvature of a peripheral edge of the tissue loading alleviation zone.

In some embodiments, as can be seen in FIG. 7B, the defined sub-regions in the tissue loading alleviation zones may represent a plurality of sub-zones, e.g., circumscribed rings and/or any other configuration, wherein each sub-zone is associated with a specified stiffness value.

In some embodiments, the tissue loading alleviation zone configurations are generated randomly within predefined ranges for each tissue loading alleviation zone parameter.

At step 402, the data representing an internal tissue loading state generated with reference to step 204 in FIG. 2 may be used to determine a total tissue loading state parameter representing a computed combination of mechanical load affecting each of the high-risk and peripheral sub-VOIs.

In some embodiments, the magnitudes and distributions of mechanical loads that are formed in the defined sub-VOIs are determined by calculating the volumetric exposures of the foot to, e.g., von Mises (effective) (σ_{vM}) and shear (σₛ) Cauchy stresses, with the von Mises stress and shear stress defined by Eq. (1) and Eq. (2), respectively:

$${σ}_{s} = max ( \frac{\vert {σ}_{1} - {σ}_{2} \vert}{2} , \frac{\vert {σ}_{2} - {σ}_{3} \vert}{2} , \frac{\vert {σ}_{3} - {σ}_{1} \vert}{2} ) \qquad ( 1 )$$

$${σ}_{v M} = \sqrt{{σ}_{x x}^{2} + {σ}_{y y}^{2} + {σ}_{z z}^{2} - {σ}_{x x} {σ}_{y y} - {σ}_{y y} {σ}_{z z} - {σ}_{z z} {σ}_{x x} + 3 ( {σ}_{x y}^{2} + {σ}_{y z}^{2} + {σ}_{z x}^{2} )} \qquad ( 2 )$$

where σₓₓ, σ_{yy}, σ_{zz} are the normal stresses in the x, y, or z directions, respectively, σ_{xy}, σ_{yz}, σ_{zx} are the shear stresses, and σ₁, σ₂, σ₃ are the principal values of the Cauchy stress tensor σ. For each model, σ_{vM} and σₛ are collected from the element in the two high-risk and peripheral sub-VOIs.

In some embodiments, a total stress-concentration exposure (TSCE) value of the entire VOI may be computed based on the values calculated for both sub-VOIs. The TSCE is defined as the area bounded between von Mises and shear stress volumetric exposure curve and the horizontal (stress) axis. To focus on exposures to elevated or focal tissue stresses, the TSCE may be calculated only for a high stress range (i.e., the upper 90% and 60% of the high-risk sub-VOI and peripheral sub-VOI stress, respectively).

In some embodiments, in order to compare between different volumes, each TSCE is normalized to the range of 0 to 100. Next, the predicted biomechanical efficacy of the support configurations is evaluated as the reduction in the TSCE value of the high-risk sub-VOI and peripheral sub-VOI. To allow comparison of TSCE across different configurations, the applied normal reaction force may be assigned to be equal in all simulations. This may be achieved by calculating the relative stresses of two subsequent FE time steps, in which the reaction force is just below and above the required force, using linear interpolation between the two steps for each mash element.

At step 404, a final tissue loading alleviation zone configuration is selected form the generated set, based on the tissue loading state analysis performed in step 402.

### EXAMPLES

In order to assess the performance of the present process, custom-made foot support structures of the present disclosure configurations were tested.

Each configuration was designed as a flat rectangular support with a round-shaped cavity in the posterior region. The offloading cavity geometry was defined in a simplified manner using three parameters: the cavity radius (X), the radius of curvature (ROC) of the cavity descent (Y), and the depth of the cavity (Z) (FIG. 7(c)). 150 samples were randomly picked within the range of 5-30 mm, 2-230 mm and 1-15 mm for the X, Y and Z parameters, respectively using Matlab's ‘rand’ function. For baseline, the case of a flat support with no cavity was examined. Solidworks® (Dassault Systèmes, CS, France) was used to design the offloading supports. Each support configuration was applied to the heel model at the pre-processing stage in PreView of FEBio (Ver. 1.19.0). The friction coefficient between the foot and the support was taken as 0.6.

TABLE 1
Mechanical properties of the model components and characteristics of the finite element mesh.

| Model | Elastic | Poisson's | | | | | Number of |
|---|---|---|---|---|---|---|---|
| component | modulus [MPa] | ratio | γ₁ | τ₁ | γ₂ | τ₂ | elements |
| Skin | 0.0954 | 0.495 | 0.0864 | 0.212 | 0.214 | 4.68 | 47,031 |
| Soft tissue | 0.306 | 0.495 | 0.3988 | 2.04 | 0.12381 | 76.96 | 132,829 |
| Achilles Tendon | 0.173 | 0.49 | — | — | — | — | 4,557 |
| Bones | 7300 | 0.3 | — | — | — | — | 68,353 |
| Cartilage | 1.01 | 0.4 | — | — | — | — | 9,861 |
| Foot supports | 10 | 0.3 | — | — | — | — | 102,532-129,878 |

The mechanical behavior and properties of all tissues were adopted from the literature (see Table 1). Specifically, the bones and AT were assumed to be isotropic linear-elastic materials with elastic moduli of 7300 MPa and 0.173 kPa, and Poisson's ratios of 0.3 and 0.49, respectively. Skin and soft tissues were assumed to behave as viscoelastic solids. The hyperelastic component of this viscoelastic behavior was considered to be the Neo-Hookean with a strain energy density function W (Eq. (3)):

$$W = \frac{μ}{2} ( {I}_{1} - 3 ) - μ ln ( J ) + \frac{1}{2} {λ ( ln J )}^{2} \qquad ( 3 )$$

where I₁ is the first invariant of the right Cauchy-Green deformation tensor, J is the determinant of the deformation gradient tensor, and μ and λ are Lamè parameters. The viscous component was simulated using the Prony-series of stress relaxation functions G (Eq. 4):

$$G ( t ) = 1 + {Σ}_{i = 1}^{2} {γ}_{i} exp ( - \frac{t}{{τ}_{i}} ) \qquad ( 4 )$$

where γᵢ and τᵢ are tissue-specific material constants, specified in Table 1 and t is time.

Ethylene-vinyl acetate (EVA) material was used for all support configurations using non-linear isotropic materials. The large deformation behavior of EVA was described using a compressible Neo-Hookean material model.

Boundary conditions were chosen to simulate the foot during heel strike on the offloading support. The initial angle between the heel and the support was approximated by 10° to simulate the foot position at heel strike. The superior surfaces of the tibia, fibula and soft tissue were fully fixed to simulate the effects of constraints from superior-lying tissues.

To simulate a ground reaction force (GRF) of 320N applied to the heel during heel-strike, displacements in the range of 7-10 mm of the flat support towards the heel were used. For calibration, the differences in the total soft tissue thickness at the bottom of the foot between the unloaded and loaded foot were used. For the upwards displacement of the flat support providing the required GRF, agreement between the resulting difference in soft tissue thickness and the difference reported in the literature during heel strike was verified.

All meshing was performed using the ScanIP+FE module of Simpleware®. All elements were of the tetrahedral type; the number of elements in each model component is specified in Table 1. The FE simulations were all set up using Pre-View of FEBio (Ver. 1.19), analyzed using the Pardiso linear solver of FEBio (Ver. 2.8.3) and post-processed using Matlab® (Math work s, Natick, MA, US) and PostView of FEBio (Ver. 2.3). The runtime of each simulation was approximately 40 minutes using a 64-bit Windows 10 based workstation with an Intel Core i7-6700 3.4 GHz CPU and 24 GB of RAM.

For each analysis, TSCE scores were computed as described in step 402.

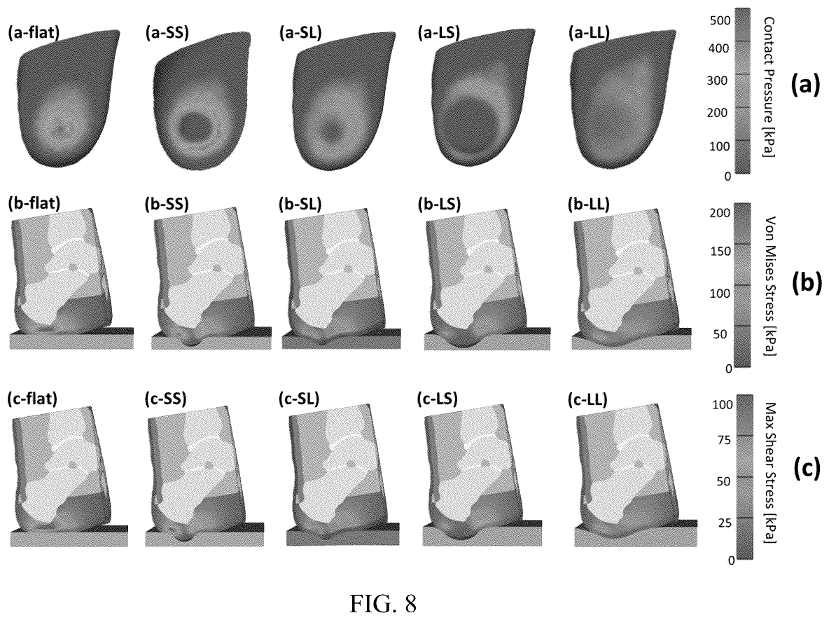

Plantar contact pressure distributions on the soft tissues with flat support and four other representative heel-support models are presented from an inferior view in FIG. 8(a). Distributions of von Mises and shear stresses are shown in cross-section views in FIG. 8(a), 8(b). Skin contact pressures were in the range of 0-500 kPa and the von Mises and shear soft tissue stresses were in the range of 0-200 kPa and 0-100 kPa, respectively. Contact pressure values agreed well with those obtained from previous computational and in-vivo experimental studies that measured interface foot pressure during gait, which thereby validates the present modelling. Contact pressure and von-Mises and shear stress clearly indicate that the geometry of the offloading cavity has great importance on the stress magnitude and distribution. Comparison of von Mises stress and shear stress in FIG. 8 shows similar distribution with no significant difference in the relative effect of the various configurations.

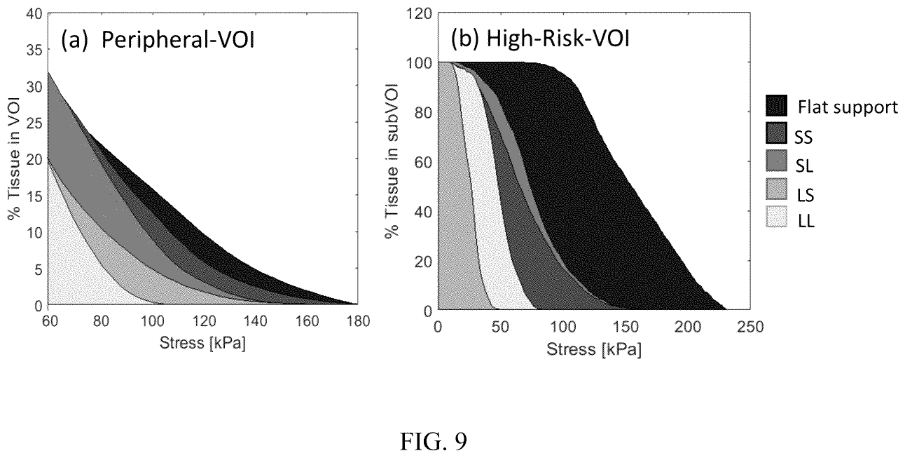

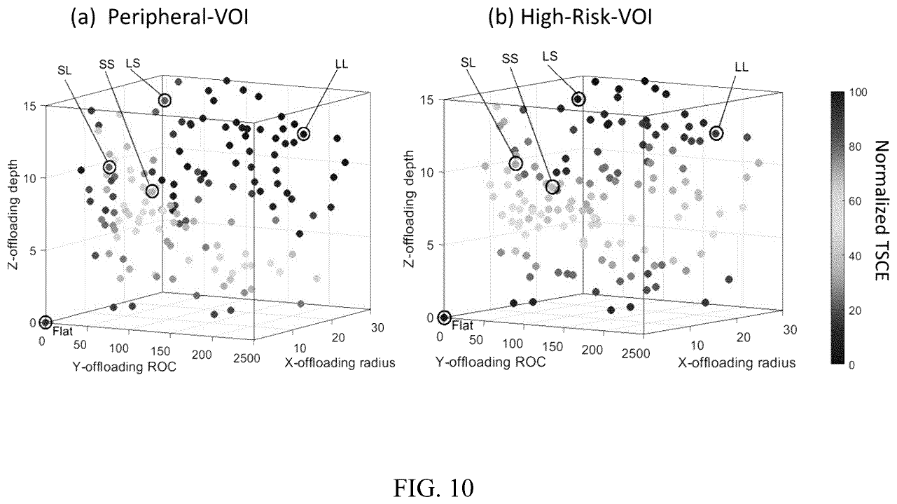

Consistent with the above results, comparison of the volumetric tissue exposures to von Mises stress in both high-risk sub-VOI and peripheral sub-VOI for five representative configurations (FIG. 9) reveals considerable variation between the different models. Values of the normalized TSCE for the 150 different offloading geometries for the high-risk sub-VOI and peripheral sub-VOI are shown in FIG. 10. Each point in the 3D space represents a different foot support configuration characterized by the three geometry parameters (X, Y, and Z axis values) previously defined (FIG. 7(C)). Point (0,0,0) represents the baseline flat support configuration. The color of the points represents the value of the TSCE, with red dots representing the high TSCE values and blue dots the low TSCE values.

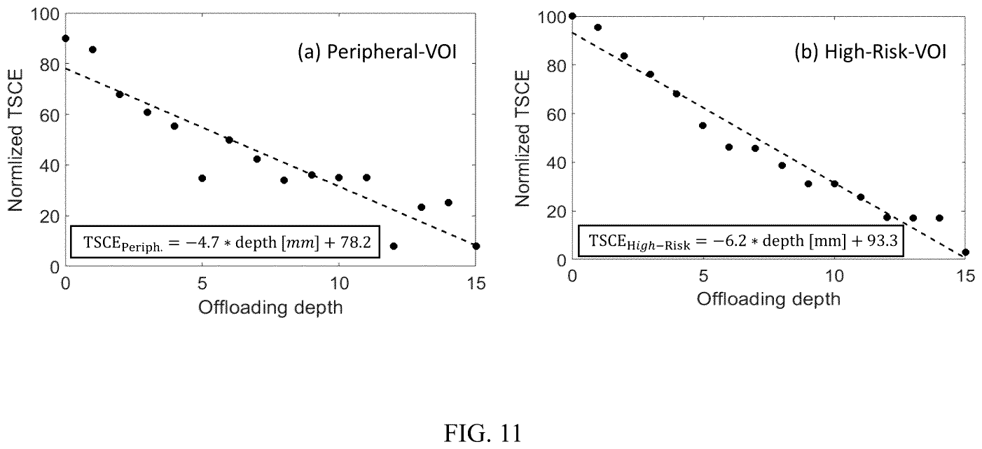

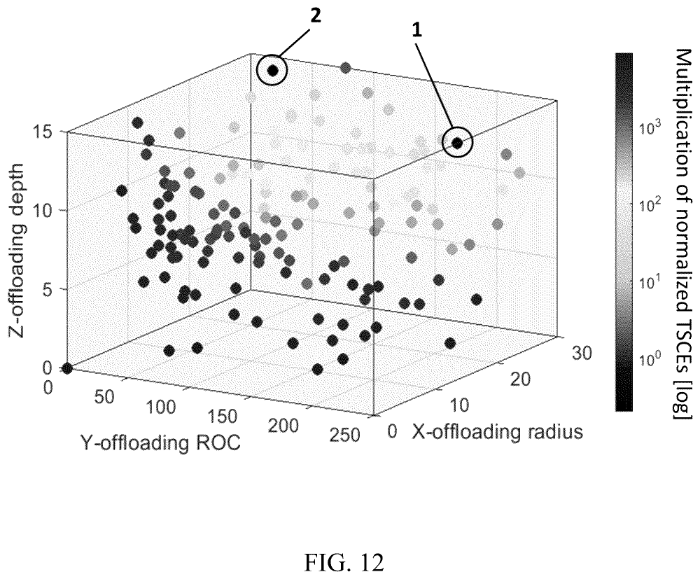

In general, volumetric exposures to stress in the peripheral sub-VOI (FIG. 11(A)) decreased with the increase of all three geometry parameters, i.e. offloading radius (X), radius of curvature (ROC) of the cavity descent (Y), and offloading depth (Z), while the high-risk sub-VOI (FIG. 11(B)) was most affected by offloading depth (Z) and less sensitive to change in X and Y. When considering the offloading depth, while averaging other parameters on each step of the Z-axis, linear behavior was identified in both high-risk and peripheral VOIs, with linear fit of 0.97 and 0.86, respectively (FIG. 12). Small cavity depth lead to high TSCE, and as the depth of the offloading cavity increases, the TSCE average value decreased linearly (FIG. 11(A,B)).

Weighted operation values of high-risk and peripheral TSCE of each configuration are shown in FIG. 12 in log scale. The two minimum values are represented by blue dots obtained at: (a) large values of all three cavity parameters (i.e. offloading radius, ROC and depth), and (b) large values of offloading radius and depth but relatively small value of ROC.

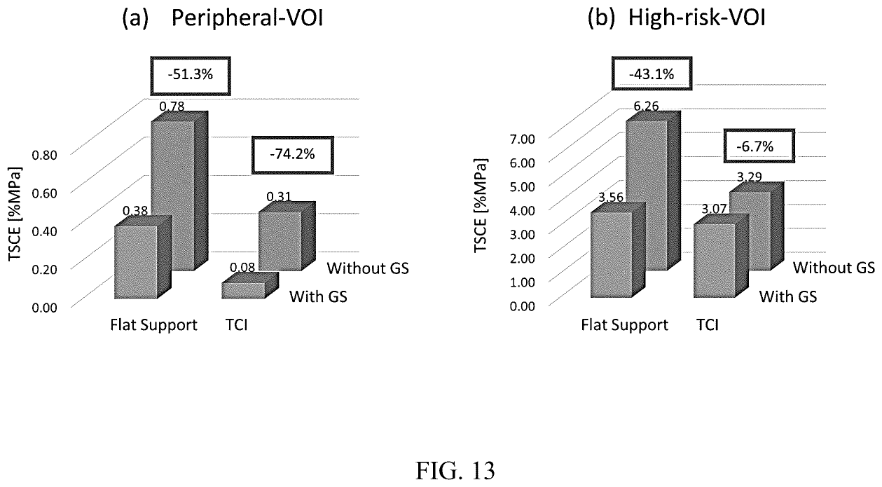

Values of the normalized TSCE scores for the two representative off-loading cavity support geometries with and without graded stiffness are shown in FIG. 13. Each graph of result (A,B) represents the VOI and sub-VOI respectively. Within each group, the lateral axis represents the insole geometry (standard or TCI) and the perpendicular axis of each group show results with and without graded stiffness. The number above each pair of result represents the percentage change from non-graded stiffness to graded stiffness configuration. Specifically, the TSCE at VOI was reduced by 51.3% and 74.2% with graded stiffness configuration at flat and with TCI configuration, respectively. At sub-VOI the TSCE was reduced by 43.1% and 6.7% with flat and TCI configurations respectively.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

Any combination of one or more computer readable medium(s) may be utilized. The computer readable medium may be a computer readable signal medium or a computer readable storage medium. A computer readable storage medium may be, for example, but not limited to, an electronic, magnetic, optical, electromagnetic, infrared, or semiconductor system, apparatus, or device, or any suitable combination of the foregoing. More specific examples (a non-exhaustive list) of the computer readable storage medium would include the following: an electrical connection having one or more wires, a portable computer diskette, a hard disk, a random access memory (RAM), a read-only memory (ROM), an erasable programmable read-only memory (EPROM or Flash memory), an optical fiber, a portable compact disc read-only memory (CD-ROM), an optical storage device, a magnetic storage device, or any suitable combination of the foregoing. In the context of this document, a computer readable storage medium may be any tangible medium that can contain or store a program for use by or in connection with an instruction execution system, apparatus, or device.

A computer readable signal medium may include a propagated data signal with computer readable program code embodied therein, for example, in baseband or as part of a carrier wave. Such a propagated signal may take any of a variety of forms, including, but not limited to, electro-magnetic, optical, or any suitable combination thereof. A computer readable signal medium may be any computer readable medium that is not a computer readable storage medium and that can communicate, propagate, or transport a program for use by or in connection with an instruction execution system, apparatus, or device.

Program code embodied on a computer readable medium may be transmitted using any appropriate medium, including but not limited to wireless, wireline, optical fiber cable, RF, etc., or any suitable combination of the foregoing.

Computer program code for carrying out operations for aspects of the present invention may be written in any combination of one or more programming languages, including an object-oriented programming language such as Java, Smalltalk, C++ or the like and conventional procedural programming languages, such as the “C” programming language or similar programming languages. The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Aspects of the present invention are described herein with reference to flowchart illustrations and/or block diagrams of methods, apparatus (systems) and computer program products according to embodiments of the invention. It will be understood that each block of the flowchart illustrations and/or block diagrams, and combinations of blocks in the flowchart illustrations and/or block diagrams, can be implemented by computer program instructions. These computer program instructions may be provided to a hardware processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable medium that can direct a computer, other programmable data processing apparatus, or other devices to function in a particular manner, such that the instructions stored in the computer readable medium produce an article of manufacture including instructions which implement the function/act specified in the flowchart and/or block diagram block or blocks.

The computer program instructions may also be loaded onto a computer, other programmable data processing apparatus, or other devices to cause a series of operational steps to be performed on the computer, other programmable apparatus or other devices to produce a computer implemented process such that the instructions which execute on the computer or other programmable apparatus provide processes for implementing the functions/acts specified in the flowchart and/or block diagram block or blocks.

The flowcharts and block diagrams in the Figures illustrate the architecture, functionality, and operation of possible implementations of systems, methods and computer program products according to various embodiments of the present invention. In this regard, each block in the flowchart or block diagrams may represent a module, segment, or portion of code, which comprises one or more executable instructions for implementing the specified logical function(s). It should also be noted that, in some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

In the description and claims of the application, each of the words “comprise” “include” and “have”, and forms thereof, are not necessarily limited to members in a list with which the words may be associated. In addition, where there are inconsistencies between this application and any document incorporated by reference, it is hereby intended that the present application controls.

1. A foot support structure comprising:
a foot support surface for engagement by a foot of a subject; and
a tissue loading alleviation zone within said foot support surface,
wherein said tissue loading alleviation zone defines a region of said foot support surface configured for optimizing an internal tissue loading state in a volume-of-interest (VOI) in said foot of said subject, and
wherein said region has at least one mechanical property value that gradually varies along at least one dimension of said region and wherein said mechanical property's graduated variation is determined based on said internal tissue loading state.

2. The foot support structure of claim 1, wherein said internal tissue loading state is determined based on one or more of: tissue strain, tissue compressive strain, tissue tensile strain, tissue shear strain, tissue strain energy density, tissue compressive stress, tissue tensile stress, tissue shear stress, and tissue hydrostatic pressure.

3. The foot support structure of claim 1, wherein said at least one mechanical property is one of: resiliency, flexibility, elasticity, density, stiffness, and compressibility.

4. The foot support structure of claim 1, wherein said region comprises a plurality of sub-zones, and wherein said at least one mechanical property value gradually varies by associating each of said sub-zones with a specified value of said at least one mechanical property.

5. The foot support structure of claim 4, wherein said optimizing comprises determining, with respect to the tissue loading alleviation zone, one or more of: dimensions of the tissue loading alleviation zone, an outline of the tissue loading alleviation zone, arrangement of said sub-zones within the tissue loading alleviation zone, a number of said sub-zones, dimensions of each of said sub-zones, and at least one mechanical property value associated with each of said sub-zones.

6. The foot support structure of claim 4, wherein said sub-zones are arranged in a sequence representing a graduated change in said at least one mechanical property along said at least one dimension of said region.

7. The foot support structure of claim 6, wherein said graduated change represents one of: an increase or a decrease in said value of said at least one mechanical property along said at least one dimension of said region.

8. The foot support structure of claim 4, comprising a plurality of sections, wherein each of said sections may be removably secured to said foot support structure, and wherein all of said sections together form said foot support surface for engagement by said foot of said subject.

9. The foot support structure of claim 8, wherein each of said sections is associated with a specified value of said at least one mechanical property.

10. The foot support structure of claim 4, wherein said plurality of sub-zones comprises a plurality of said sections arranged to create said tissue loading alleviation zone based, at least in part, on said associated at least one mechanical property of each of said sections.

11-13. (canceled)

14. The foot support structure of claim 1, wherein said VOI encompasses at least one of: an ulcerated region of said foot, a region of said foot representing ulceration risk, and a peripheral region surrounding an ulcerated region of said foot.

15.-19. (canceled)

20. A method comprising:
receiving, as input, a volumetric scan of an anatomy of a foot of a subject;
generating data representing an internal tissue loading state of a volume of interest (VOI) in said foot, based on the scan;
defining, in said VOI, a high-risk sub-VOI and a peripheral sub-VOI; and
calculating, based on said data, a set of parameters for a tissue loading alleviation zone within a foot support surface of a foot support structure for engagement by said foot of said subject, wherein said calculation minimizes a combined tissue loading index in said high-risk and peripheral sub-VOIs.

21-25. (canceled)

26. The method of claim 20, wherein said data representing an internal tissue loading state takes into account mechanical properties of at least some of: foot bone tissue, foot cartilage, foot tendons, foot soft tissue, and foot skin within said VOI.

27. The method of claim 20, wherein, with respect to each of said high-risk sub-VOI and peripheral sub-VOI, said tissue loading represents one or more of: tissue strain, tissue compressive strain, tissue tensile strain, tissue shear strain, tissue strain energy density, tissue compressive stress, tissue tensile stress, tissue shear stress, and tissue hydrostatic pressure.

28. The method of claim 20, wherein said tissue loading alleviation zone represents a downward concavity in said pressure support surface, and wherein said set of parameters comprises one or more of:
(i) dimensions of said tissue loading alleviation zone;
(ii) an outline of a peripheral edge of said tissue loading alleviation zone;
(iii) a total surface area defined by said peripheral edge;
(iv) a depth of said tissue loading alleviation zone; and
(v) a radius of curvature of said peripheral edge.

29. The method of claim 28, wherein said outline is circular, and said set of parameters comprises a diameter of said outline.

30. The method of claim 20, wherein said set of parameters gradually varies at least one mechanical property value along at least one dimension of a region defined by said tissue loading alleviation zone.

31. The method of claim 30, wherein said at least one mechanical property is one of: resiliency, flexibility, elasticity, density, stiffness, and compressibility.

32. The method of claim 30, wherein said region comprises a plurality of sub-zones, and wherein said at least one mechanical property value gradually varies by associating each of said sub-zones with a specified value of said at least one mechanical property.

33. The method of claim 32, wherein said sub-zones are arranged in a sequence representing a graduated change in said at least one mechanical property along said at least one dimension of said region.

34-43. (canceled)
